(19) United States
(12) Patent Application Publication
(10) Pub. No.: US 2007/0142903 A1
(43) Pub. Date: Jun. 21, 2007

(54) Laser cut intraluminal medical devices

(76) Inventor: Vipul Bhupendra Dave, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/304,372

(22) Filed: Dec. 15, 2005

(51) Int. Cl. A61F 2/88; B23K 26/38; B23K 26/12

(52) U.S. Cl. 623/1.22; 623/1.42; 424/426; 219/121.72

(57) ABSTRACT

Laser cut bioabsorbable intraluminal devices or stents and methods for forming such an intraluminal device or stent. A precursor sheet or tube of bioabsorbable material is laser cut in the presence of an inert gas to form an intraluminal medical device or stent having a desired geometry or pattern. The device or stent may comprise a helical, or other shape, having the laser cut geometry or pattern imparted thereon. The device or stent may further comprise drugs or bio-active agents incorporated into or onto the device or stent in greater percentages than conventional devices or stents. Radiopaque materials may be incorporated into, or coated onto, the intraluminal device or stent. Precise geometries or patterns are simply and readily achievable using the laser cutting methods in the presence of an inert gas while minimizing damage to the precursor materials.

### BACKGROUND OF THE INVENTION

1. Field of the Invention

The invention generally relates to bioabsorbable intraluminal medical devices that are laser cut in an inert gas atmosphere to impart a desired geometry or pattern to the device.

2. Related Art

Intraluminal endovascular medical devices, such as stents, are well-known. Such stents are often used for repairing blood vessels narrowed or occluded by disease, for example, or for use within other body passageways or ducts. Typically the stent is percutaneously routed to a treatment site and is expanded to maintain or restore the patency of the blood vessel or other passageway or duct within which the stent is emplaced. The stent may be a self-expanding stent comprised of materials that expand after insertion according to the body temperature of the patient, or the stent may be independently expandable by an outwardly directed radial force from a balloon, for example, whereby the force from the balloon is exerted on an inner surface of the stent to expand the stent towards an inner surface of the vessel or other passageway or duct within which the stent is placed. Ideally, once placed within the vessel or other passageway or duct, the stent will conform to the contours and functions of the blood vessel or other body passageway in which the stent is deployed.

Moreover, as in U.S. Pat. No. 5,464,450, stents are known to have been comprised of biodegradable materials, whereby the main body of the stent degrades in a predictably controlled manner. Stents of this type may further comprise drugs or other biologically active agents that are contained within the biodegradable materials. Thus, the drugs or other agents are released as the biodegradable materials of the stent degrade.

Although such drug containing biodegradable stents as described in U.S. Pat. No. 5,464,450 may be formed by mixing or solubilizing the drugs with the biodegradable polymer comprising the stent, by dispersing the drug into the polymer during extrusion of the polymer, or by coating the drug onto an already formed film or fiber, such stents typically include relatively small amounts of drugs. For example, U.S. Pat. No. 5,464,450 contemplates containing only up to 5% aspirin or heparin in its stent for delivery therefrom.

Further, such stents, as disclosed in U.S. Pat. No. 4,464,450, are often made without radiopaque markers. The omission of radiopaque markers inhibits the visualization and accurate placement of the stent by the medical practitioner.

Polymers are often processed in melt conditions and at temperatures that may be higher than is conducive to the stability of the drugs or other agents to be incorporated into a bioabsorbable drug delivery device. Typical methods of preparing biodegradable polymeric drugs delivery devices such as stents include fiber spinning, film or tube extrusion, or injection molding. All of these methods tend to use processing temperatures that are higher than the melting temperature of the polymers. Processing at such conditions tends to compromise the physical properties of the materials comprising the stent. Moreover, most bioabsorbable polymers melt process at temperatures higher than 130°-160° C., which represent temperatures at which most drugs are not stable and tend to degrade.

Stents of different geometries are also known. For example, stents such as disclosed in U.S. Pat. No. 6,423,091 are known to comprise a helical pattern comprised of a tubular member having a plurality of longitudinal struts with opposed ends.

None of the various art described combines techniques to provide a bioabsorbable intraluminal medical device, such as a stent, that is formed using mask projection laser cutting techniques to provide an intraluminal device or stent of desired geometries or patterns having increased drug delivery capacity and radiopacity while minimizing damage to the materials comprising the device or stent during processing.

In view of the above, a need exists for systems and methods that form implantable bioabsorbable polymeric drug delivery devices with desired geometries or patterns, wherein the devices have increased and more effective drug delivery capacity and radiopacity. Further in view of the above, a need exists for systems and methods that simplify the machining and formation of such laser cut bioabsorbable intraluminal devices or stents.

### SUMMARY OF THE INVENTION

The systems and methods of the invention provide a bioabsorbable intraluminal device or stent that is implantable within the vasculature or other passageway of a patient. The intraluminal device or stent is laser cut in an inert gas atmosphere into desired geometries or patterns. The device or stent is formed into an appropriate shape, such as a helical, or other, shape, conducive to emplacement in a vessel or other anatomical passageway of a patient. The techniques of laser cutting a precursor material in the presence of the inert gas renders precise geometries or patterns more simply and readily achievable, ideally, without compromising the strength or endurance of the intraluminal device or stent. The device or stent preferably further comprises drugs or other bio-active agents incorporated into or applied onto the device or stent in greater percentages than commonly provided in conventional devices or stents. Radiopaque material may further comprise the intraluminal devices or stents, wherein such radiopaque material is incorporated into or applied onto the materials comprising the device or stent. The drugs, bio-active agents or radiopaque materials may be provided before or after laser cutting of the precursor material and formation of the device or stent occurs.

In some embodiments of the systems and methods of the invention, the materials from which the intraluminal device or stent is made are provided from a precursor sheet of bioabsorbable materials, wherein the desired geometry or pattern is laser cut into the precursor sheet and the sheet is then wound into a helical, or other, shape. The precursor sheet is produced from conventional compression molding or solvent casting techniques, for example.

In other embodiments of the systems and methods of the invention, the materials from which the intraluminal device or stent is made are provided from a precursor tube of bioabsorable materials. The precursor tube is produced from conventional melt extrusion and solvent-based processes, for example. The desired geometry or pattern is thus laser cut into the precursor tube.

In practice, the precursor sheet or tube of bioabsorbable material is mounted to a laser processing unit and subjected to energy from a laser beam in order to form an implantable device or stent having the desired geometry or pattern imparted thereon. An inert gas is provided within the atmosphere in which the laser cutting occurs. A mask, having the desired geometry or pattern ultimately imparted to the device or stent, is provided above the bioabsorbable material and the laser beam to help impart the intended geometry or pattern to the precursor material by the laser beam. The laser processing unit preferably comprises a co-ordinated multi-motion unit that moves the laser beam in one direction and the material in another direction when subjecting the material to the laser beam for cutting thereof the precursor material. The laser beam is projected through the mask and ablates the bioabsorbable material, thus imparting to the device or stent the geometry or pattern corresponding to the mask. Inert gas provided in the laser-cutting environment minimizes, or ideally eliminates, moisture and oxygen related effects during laser cutting of the material.

Preferably, the laser beam is further directed through a lens before reaching the precursor material. The lens intensifies the beam and more precisely imparts the desired pattern or geometry onto the materials. A beam homogenizer may also be used to create more uniform laser beam energy and to maintain the laser beam energy consistency as the beam strikes the material. In this way, laser-machined features are more simply and readily achieved in the desired geometry or pattern. Beam energy can be controlled to reduce the laser cutting time.

After laser cutting the desired geometry or pattern onto the precursor material, the precursor material is removed from the laser cutting unit and stored until needed, in the case of the tube, or formed into the desired shaped, i.e., helical or otherwise, and then stored until needed. Precursor materials of various dimensions may thus be laser-cut using the techniques described herein in order to provide intraluminal medical devices or stents having various axial and radial strength and flexibility, or other characteristics, to better suit various medical and physiological needs. The geometries or patterns imparted to the precursor material can comprise helical, non-helical, or combinations thereof, that extend over all, some or at discrete intervals of the length of the device or stent ultimately formed.

The above and other features of the invention, including various novel details of construction and combinations of parts, will now be more particularly described with reference to the accompanying drawings and claims. It will be understood that the various exemplary embodiments of the invention described herein are shown by way of illustration only and not as a limitation thereof. The principles and features of this invention may be employed in various alternative embodiments without departing from the scope of the invention.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 illustrates a precursor sheet of bioabsorbable material according to the systems and methods of the invention.

FIG. 2 illustrates a precursor tube of bioabsorbable material according to the systems and methods of the invention.

FIG. 3 illustrates a laser processing unit for laser cutting the precursor sheet of FIG. 1 or the precursor tube of FIG. 2 according to the systems and methods of the invention.

FIGS. 4 illustrates a partial view of the laser processing unit of FIG. 3 including a mask through which the laser beam penetrates to impart a geometry or pattern onto a precursor sheet or tube according to the systems and methods of the invention.

FIGS. 5A-5C illustrates portions of helical coiled stents having a geometry or pattern laser cut from a precursor sheet according to the systems and methods of the invention.

FIG. 6 illustrates portions of a stent having a geometry or pattern laser cut from a precursor tube according to the systems and methods of the invention.

FIGS. 7A-7C illustrate stents having other geometries or patterns laser cut from a precursor tube according to the systems and methods of the invention.

FIGS. 8A-8C illustrate various other geometries and patterns laser cut from a precursor material according to the systems and methods of the invention.

### DETAILED DESCRIPTION OF THE INVENTION

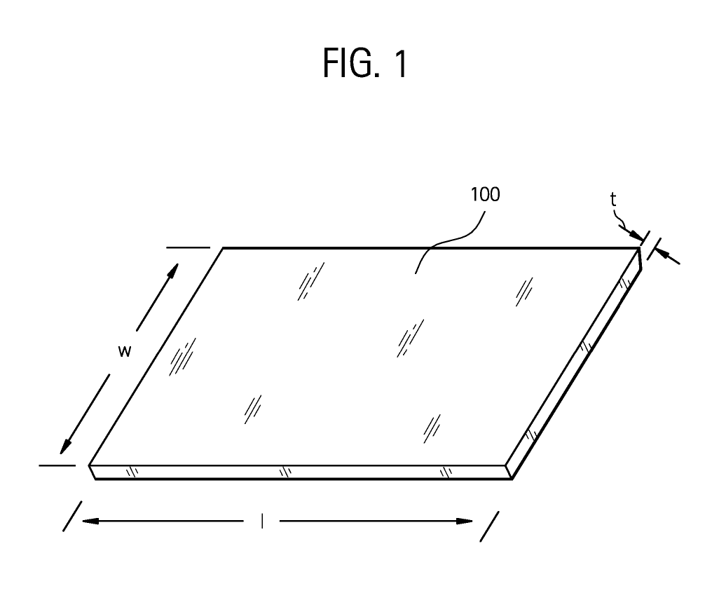

FIG. 1 illustrates a precursor sheet 100 of bioabsorbable material for forming an intraluminal medical device or stent according to the systems and methods of the invention. The precursor sheet 100 is produced from conventional compression molding or solvent casting techniques, for example, which are not further detailed herein as the artisan should readily appreciate how such precursor sheets 100 are formed using conventional techniques. The precursor sheet 100 is provided with length (l), width (w) and thickness (t) dimensions that may be varied from sheet to sheet in order to accommodate the formation of differently sized medical devices or stents. For example, where a longer anatomic vessel or passageway is the intended treatment site, then a longer length (l) dimension may be provided, or where increased radial strength is desirable, then a larger thickness (t) dimension may be provided. The precursor sheet 100 is comprised of bioabsorbable materials such as, for example, aliphatic polyesters (polylactic acid; polyglycolic acid; polycaprolactone; polydioxanone; poly (trimethylene carbonate), poly (oxaesters), poly (oxaamides), and their co-polymers and blends; poly(anhydrides) includes poly(carboxyphenoxy hexane-sebacicacid), poly (fumaric acid-sebacic acid), poly (carboxyphenoxy hexane-sebacic acid), poly (imide-sebacic acid) (50-50), and poly (imide-carboxyphenoxy hexane) (33-67), poly (orthoesters) (diketene acetal based polymers); tyrosine derived poly amino acid [examples: poly (DTH carbonates), poly (arylates), and poly (imino-carbonates) ], phosphorous containing polymers [examples: poly(phosphoesters) and poly (phosphazenes)], poly (ethylene glycol) [PEG] based block co-polymers [PEG-PLA, PEG-poly (propylene glycol), PEG-poly (butylenes terphthalate)], poly (α-malic acid), poly (ester amide), and polyalkanoates [examples: poly (hydroxybutyrate (HB) and poly (hydroxyvalerate) (HV) co-polymers].

Of course, the artisan will appreciate that other known or later developed bioabsorbable materials conducive to implantation within the vasculature or anatomical passageways of a patient are contemplated for comprising the medical device or stent formed according to the systems and methods according to the invention as well. The bioabsorbable materials comprising the precursor sheet 100, and the dimensions thereof, contribute to the axial and radial strength, and flexibility, characteristics of the device or stent.

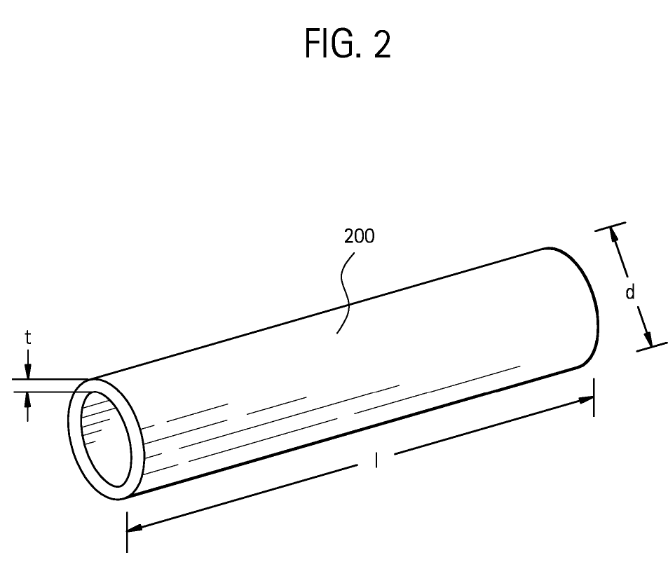

FIG. 2 illustrates a precursor tube 200 of bioabsorbable material according to the systems and methods of the invention. The precursor tube 200 is produced from conventional melt extrusion and solvent-based processing techniques, for example, which are not further detailed herein as the artisan should readily appreciate how such precursor tubes 200 are formed using conventional techniques. The precursor tube 200 is provided with length (l), diameter (d) and thickness (t) dimensions that may be varied from tube to tube in order to accommodate the formation of differently sized medical devices or stents. The precursor tubes 200 are preferably comprised of bioabsorbable materials, such as those described above with respect to the precursor sheets 100, which materials, and the dimensions thereof, contribute to the axial and radial strength, and flexibility, characteristics of the device or stent.

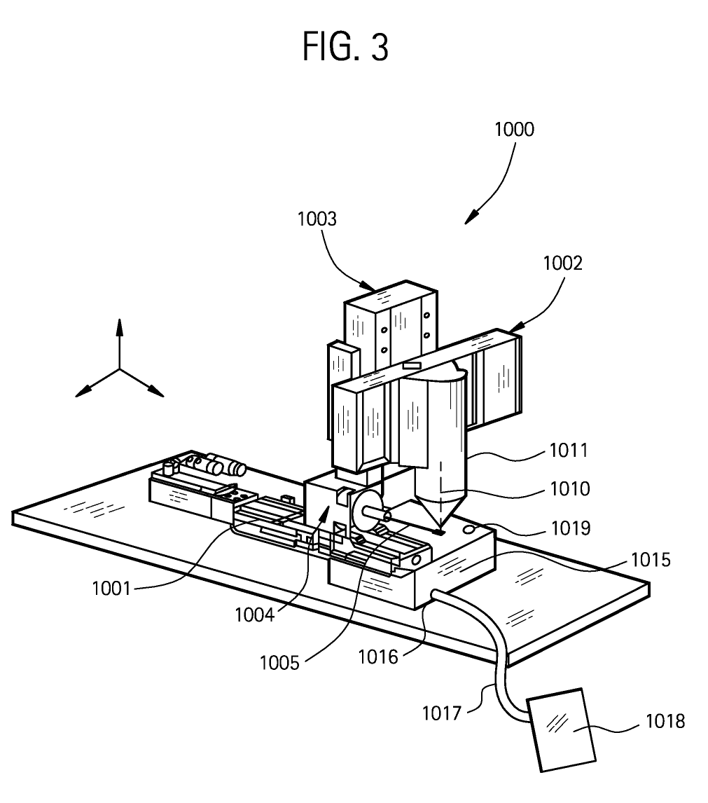

FIG. 3 illustrates a laser processing unit 1000 for laser cutting precursor material according to the systems and methods of the invention. The precursor material is either the precursor sheet 100 of FIG. 1 or the precursor tube 200 of FIG. 2. The laser processing unit 1000, which is a non-limiting example of a laser processing unit for laser cutting precursor material according to the various embodiments described herein, comprises an X-stage 1001, a Y-stage 1002, and a Z-stage 1003, wherein each stage is independently movable relative to one another. A laser beam 1010, shown in dashed lines in FIG. 3, is provided within housing 1011, for example, which is fixed to at least one of the X-stage 1001, Y-stage 1002, and Z-stage 1003. FIG. 3 illustrates the housing 1011 as fixed to the Y-stage 1002, for example, wherein the laser beam 1010 is housed therein. In practice, the precursor sheet 100 is thus arranged on the X-stage 1001 below the movement range of the laser beam 1010. Where a precursor tube 200 is used, the laser processing unit 1000 further comprises a rotary stage 1004 having a mandrel 1005 extending therefrom. In practice, the precursor tube 200 is thus arranged onto the mandrel 1005 below the movement range of the laser beam 1010, wherein the rotary stage 1004 and mandrel 1005 independently rotates the precursor tube 200 mounted thereon. Thus, where a flat precursor sheet 100 is used, the rotary stage 1004 and mandrel 1005 of FIG. 3 may be omitted, and the precursor sheet 100 is positioned along the X-stage 1001. In either case, the laser beam 1010 is moved relative to the precursor material, and preferably the precursor material is also moved relative to the laser beam 1010, so as to direct energy from the laser beam onto the precursor material.

As illustrated in FIG. 3, the laser processing unit 1000 further comprises an inert gas box 1015 that surrounds the precursor material (sheet 100/FIG. 1 or tube 200/FIG. 2) during the laser cutting process. The inert gas box 1015 includes an inlet 1016 and an outlet 1019 through which the flow of inert gas respectively enters and exits the inert gas box 1015. The inlet 1016 may be further connected to an inert gas supply 1018 via a hose 1017 or other means for supplying the inert gas to the inert gas box 1015. The inert helps minimize, or ideally eliminate, undesirable blemishes or other defects in the precursor material that is subjected to the laser cutting techniques described herein. The inert gas may be, for example, nitrogen. The artisan will readily appreciated that other laser processing units may be differently configured, while comprising the same features described herein, wherein the laser beam is moved relative to the precursor material, and preferably the precursor material is also moved relative to the laser beam.

As shown in FIG. 3, the Y-stage 1002 is shown as having the laser beam 1010 arranged therewith within housing 1010, although the artisan should readily appreciate that any, or all, of the other stages could also have a laser beam attached thereto, or omitted therefrom, so long as at least one laser beam is provided. Further, although the laser processing unit 1000 shown in FIG. 3 illustrates a unit having movement in three directions, i.e., the x, y and z directions, the artisan should appreciate that laser processing units having other directional motion capacities are also contemplated for making devices according to the systems and methods of the invention. For example, a 6-axis Co-Ordinated Motion laser processing unit may be employed whereby the precursor material is moved in one direction whereas the laser beam is moved in an opposite direction in order to impart the intended geometry or pattern to the material.

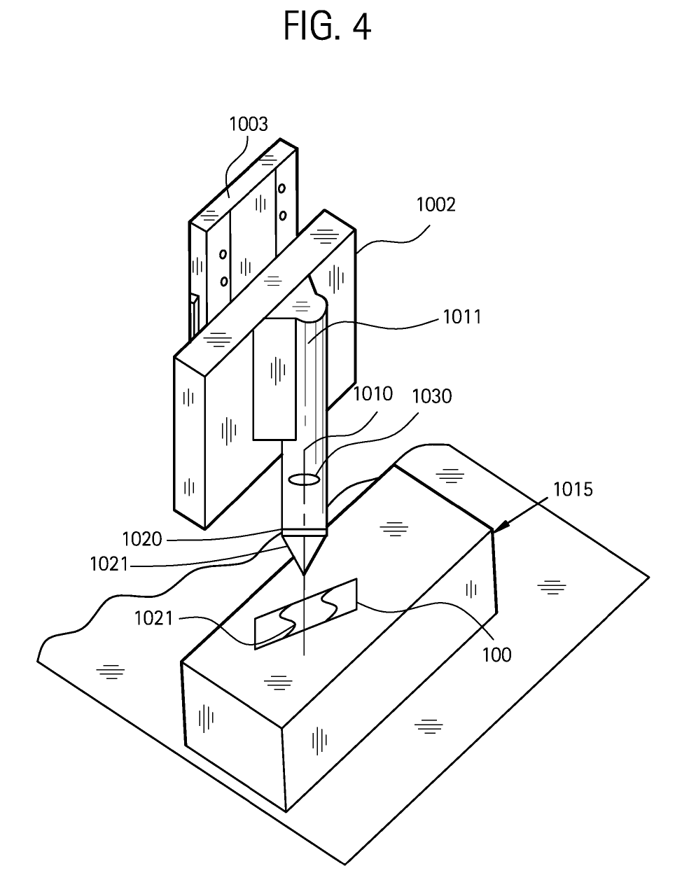

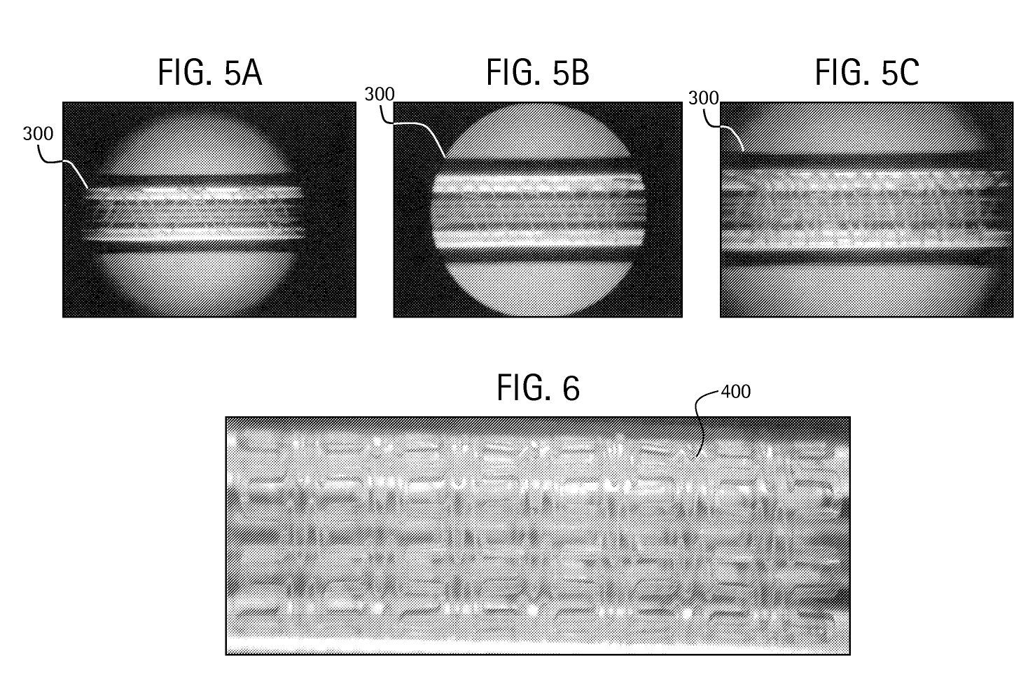

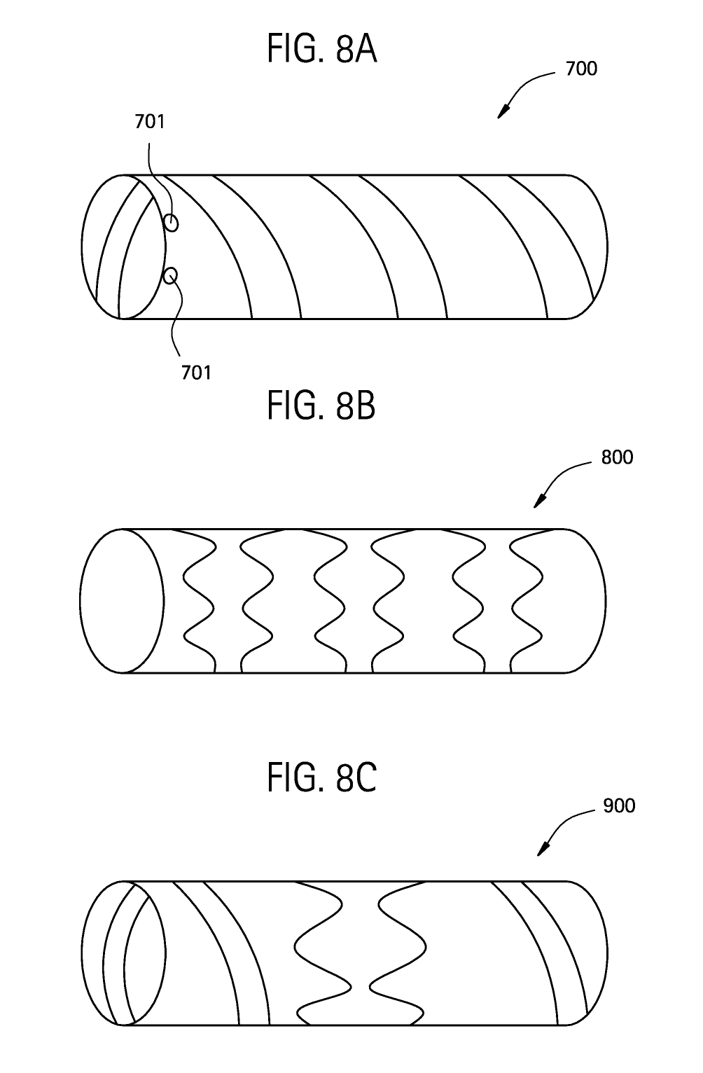

FIG. 4 illustrates a partial view of the Y-stage 1002 of the laser processing unit 1000 of FIG. 3 having a flat precursor sheet 100 arranged thereunder for laser cutting. Y-stage 1002 in this instance comprises the housing 1011 in which the laser beam 1010 (dashed lines) is arranged. The housing 1011 further comprises a lens 1030 and a mask 1020 arranged therein, through which lens 1030 and mask 1020 the laser beam 1010 projects in order to impart a geometry or pattern onto the precursor material, such as a precursor sheet 100 or tube 200. In particular, the mask 1020 includes the geometry or pattern 1021 imparted to the underlying precursor sheet 100 or tube 200 when the laser beam 1010 is projected through the mask 1020 and onto the precursor material. Although shown in FIG. 4 as having a geometry or pattern 1021 of a series of generally longitudinally adjacent segments, the artisan will readily appreciate that the geometry or pattern 1021 imparted to the precursor material is alterable to suit various medical and physiological needs. Accordingly, changing of the mask 1020 to one having a different geometry or pattern imparts the different geometry or pattern to the precursor material, wherein a uniform geometry or pattern is imparted to the precursor material or different geometries or patterns may be imparted to the precursor material. FIGS. 5-8C illustrate various non-limiting geometries or patterns 1021 impartable to precursor materials to comprise devices or stents according to the various embodiments described herein. Other known or later developed geometries or patterns conducive to emplacement and compatibility within the vasculature or other anatomical passageway of a patient may be laser cut from a precursor material to form a device or stent as otherwise described herein, including exclusively helical designs 700 (FIG. 8A), non-helical designs 800 (FIG. 8B) having one or more longitudinally adjacent segments, or combinations thereof 900 (FIG. 8C). The designs may extend the entire length of the device or stent when formed after laser cutting thereof, or may extend only partially along the length of the device or stent after laser cutting thereof, or may extend at discrete intervals along the length of the device or stent after laser cutting thereof.

Preferably, as also shown in FIG. 4, the laser processing unit 1000 further comprises a lens 1030 through which the laser beam passes in order to intensify the energy of the beam 1010 and to shrink or concentrate the geometry or pattern onto the targeted precursor material. Although FIG. 4 illustrates the lens 1030 positioned above the mask 1020, the artisan will appreciate that the lens 1030 could alternatively be positioned below the mask 1020, in order to intensify the energy of the beam 1010 as it strikes the precursor material. Three-dimensional machining of devices or stents having precision oriented geometries or patterns is simplified as a result of imparting the geometries or patterns thereto using the laser processing techniques described herein.

Although not shown, a beam homogenizer may also be used to create more uniform laser beam energy density applied to the targeted precursor material, and, ideally, to achieve more consistently machined features in the device or stent. In this respect, the laser beam 1010 is thus shaped prior to reaching the mask 1020, which can help optimize throughput of the designed device or stent.

In practice, typical conditions used to prepare the device or stent according to the systems and methods of the invention include projecting a laser beam 1010 through the lens 1030, (the beam homogenizer if provided), and the mask 1020 at a wavelength of 193 nm with an energy density of 580-600 mJ/cm2, wherein the laser repetition rate is within the range of 80-175 Hz, and the number of laser pulses is within the range of 390-1000. The 193 nm wavelength tends to provide cleaner edges with reduced thermal damage to the underlying precursor materials. The 193 nm wavelength also tends to provide higher resolutions that more readily accommodate imparting more intricate designs, geometries or patterns to the stent or device than does standard, or longer, wavelengths. Inert gas, such as nitrogen, is used in the laser-cutting atmosphere in order to minimize, or ideally, eliminate moisture and oxygen related effects during laser cutting.

According to the various embodiments described herein, a precursor polymeric material is thus converted into a device or stent by laser cutting, for example by excimer laser cutting, or micro-machining, the precursor material, in the presence of an inert gas while minimizing damage to the physical properties of the precursor material. Performing the laser cutting of the precursor material in the presence of the inert gas tends to minimize undesirable damage to the precursor material during processing as compared to other methods such as injection molding, extrusion, or other conventional techniques. Moreover, the laser cutting techniques described herein are relatively short in duration, for example 2-3 minutes, and simple to perform as compared to more conventional techniques. Flat precursors (FIG. 1) tend to take even less time to process as compared to tubular precursors (FIG. 2), although laser cutting either precursor, i.e., a flat precursor or a tubular precursor, according to the systems and methods described herein tend to take less time (2-3 minutes) than conventional techniques (typically about 5-15 minutes). Moreover, the energy of the laser beam can be controlled to vary laser cutting time. For example, laser beam energy can be raised to decrease laser cutting time, laser beam energy can be lowered to increase laser cutting time, the lens strength or orientation can be altered or the materials can be altered to control laser cutting time.

Still further, the devices or stents made in accord with the various embodiments described herein contain drugs or other bio-active agents in greater percentages by weight than conventional drug-coated metal stents. For example, the devices or stents made according to the various embodiments described herein may comprise drugs or bio-active agents in a range between 1-50% by weight, and preferably between 10-30% by weight. The drugs or other bio-active agents may be incorporated into or applied onto the precursor material prior to laser cutting, or may be incorporated into or applied onto the device or stent after laser cutting and formation thereof has occurred. Ideally, the drug content provided in the devices or stents made in accord with the embodiments described herein remains and is substantially unaffected by the laser cutting thereof.

Such drugs or other bio-active agents may be, for example, therapeutic and pharmaceutic agents including: anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagines); antiplatelet agents such as G(GP) 11b/111a inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes—dacarbazinine (DTIC); anti-proliferative/antimitotic antimetabolites such as folic acid analogs (methotrexate), pyrimidine analogs (fluorouracil, floxuridine and cytarabine) purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory; such as adrenocortical steroids (cortisol, cortisone, fludrocortisone, prednisone, prednisolone, 6a-methylprednisolone, triamcinolone, betamethasone, and dexamethasone), non-steroidal agents (salicylic acid derivatives i.e. aspirin; para-aminophenol derivatives i.e. acetaminophen; indole and indene acetic acids (indomethacin, sulindac, and etodalec), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors, antisense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

Radiopaque marker materials may also be incorporated into or applied onto some or all of the precursor material before laser cutting, or may be incorporated into or applied onto some or all of the device or stent after laser cutting and formation thereof has occurred. The radiopaque material should be biocompatible with the tissue in which the device is deployed. Such biocompatibility minimizes the likelihood of undesirable tissue reactions with the device. The radiopaque additives can include metal powders such as tantalum or gold, or metal alloys having gold, platinum, iridium, palladium, rhodium, a combination thereof, or other materials known in the art. Other radiopaque materials include barium sulfate (BaSO4); bismuth subcarbonate ((Bio)2CO3); bismuth oxides and/or iodine compounds. Ideally, the radiopaque materials should preferably adhere well to the device such that peeling or delamination of the radiopaque material from the device is minimized, or ideally does not occur.

Where the radiopaque materials are added to the device as metal bands, the metal bands may be crimped at designated sections of the device. Alternatively, designated sections of the device may be coated with a radiopaque metal powder, whereas other portions of the device are free from the metal powder. Still further alternatively, sections of the device may be laser cut into a cavity 701, FIG. 8A, for example, that is subsequently filled with radiopaque material. Of course, the cavity 701 may be made at locations or in shapes other than as shown, and may be made a part of any of the various device or stent designs described herein. As the artisan should appreciate, barium is often used as the metallic element for visualizing the device using these techniques, although tungsten and other fillers are also becoming more prevalent. The particle size of the radiopaque materials can range from nanometers to microns, and the amount of radiopaque materials can range from 1-50% (wt %).

FIGS. 5A-5C illustrate portions of helical coiled stents 300 having a geometry or pattern laser cut from a precursor sheet of bioabsorbable material according to the various embodiments described herein. FIGS. 5A-5C demonstrate stents of different dimensions or materials having varying radial strength characteristics. For example, the helical coiled stents 300 were laser cut in the presence of an inert gas using a laser processing unit such as the laser processing unit 1000 of FIGS. 3 & 4. After cutting, the precursor material is removed from the laser processing unit and wound about a mandrel, or otherwise manipulated, to form the helical shape. The radial strength of the stents of FIGS. 5A-5C ranged from 2 psi to 30 psi, depending on the thickness of the precursor material used and the pitch of the geometry or pattern imparted to the stents.

FIGS. 5A-5C illustrate portions of helical stents 300 of varying length dimensions and the same diameter, wherein each was formed from different combinations of bioabsorbable materials. For example, FIG. 5A illustrates a helical coiled stent 300 with a length of 18 mm and a 3.5 mm inner diameter; FIG. 5B illustrates a helical stent 300 with a length of 10 mm and a 3.5 mm inner diameter; and FIG. 5C illustrates a helical stent 300 with a length of 18 mm and a 3.5 mm inner diameter. Various bioabsorbable materials comprising PLLA, PLGA (95/5), PLGA (85/15) and PCL/PGA (35/65) were used to comprise the stents. Based on FIGS. 5A-5C, stents comprised of PLLA and PLGA tended to have better radial strength than the other trial materials, regardless of the length dimensions of the stent or device. Of course, the dimensions identified above can vary and may expand according to physiologic needs.

FIG. 6 illustrates another stent 400 made according to the laser processing techniques of the invention, whereby the stent 400 is fabricated from a precursor of bioabsorbable material. FIG. 6 illustrates, for example, a stent 400 having a Bx VELOCITY®, (stent) design with an 18 mm length and a range of 1-4 mm inner diameter. Precursor material thicknesses varied from 3 mils to 10 mils, and various bioabsorbable materials, for example, PLLA, PLLA/TMC Blend, PLLA/PCL Blend, PCL/PGA (35/65) and PLDL, were used. Based on FIG. 6, stents comprised of PLLA and PLDL tended to have better radial strength than the other trial materials, regardless of the thickness dimensions of the precursor materials of the stent or device. Of course, the dimensions identified above can vary and may expand according to physiologic needs.

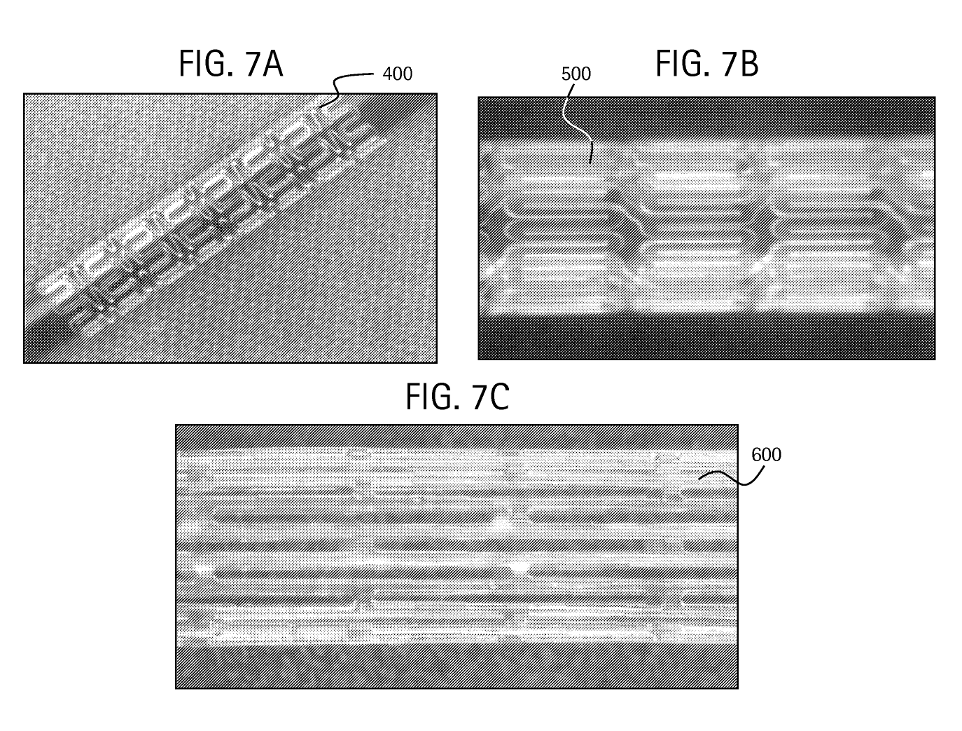

FIGS. 7A-7C illustrate various other non-limiting s examples of geometries or patterns impartable to a precursor to form a device or stent according to the systems and methods of the invention. FIG. 7A illustrates a stent 400 having a Bx VELOCITY® (stent) design; FIG. 7B illustrates a stent 500 having a S.M.A.R.T.® (stent) design; and FIG. 7C illustrates a stent 600 having a PALMAZ® (stent)design. Of course, dimensions can vary and may expand according to physiologic needs.

The various exemplary embodiments of the invention as described hereinabove do not limit different embodiments of the systems and methods of the invention. The material described herein is not limited to the materials, designs or shapes referenced herein for illustrative purposes only, and may comprise various other materials, designs or shapes suitable for the systems and methods described herein, as should be appreciated by the artisan.

While there has been shown and described what is considered to be preferred embodiments of the invention, it will, of course, be understood that various modifications and changes in form or detail could readily be made without departing from the spirit or scope of the invention. It is therefore intended that the invention be not limited to the exact forms described and illustrated herein, but should be construed to cover all modifications that may fall within the scope of the appended claims.

What is claimed is:

1. A method for forming a laser cut intraluminal device using a co-ordinated motion laser processing unit, the method comprising:
providing a precursor material;
arranging the precursor material relative to the laser processing unit;
subjecting the precursor material to energy from a laser beam in the
presence of an inert gas;
imparting a geometry and pattern to the precursor material; and
removing the precursor material from the arrangement with the laser processing unit.

2. The method of claim 2, further comprising:
providing a mask with the co-ordinated motion laser processing unit, whereby the laser beam projects through the mask to impart the geometry and pattern to the precursor material.

3. The method of claim 2, wherein providing the precursor material comprises providing a bioabsorbable material.

4. The method of claim 1, further comprising providing a lens with the laser processing unit, through which lens the laser beam passes to intensify the energy of the laser beam directed to the precursor material.

5. The method of claim 4, further comprising providing a beam homogenizer and shaping the laser beam prior to the laser beam projecting through the mask and to the precursor material.

6. The method of claim 4, further comprising projecting the laser beam through the mask and to the precursor material at a wavelength of 193 nm, with an energy density of 580-600 mJ/cm2 to impart the geometry and pattern to the precursor material.

7. The method of claim 6, further comprising a laser repetition rate of approximately 80-175 Hz, and a number of laser pulses of about 390-1000 to impart the geometry and pattern to the precursor material.

8. The method of claim 1, further comprising minimizing moisture and oxygen effects during laser cutting of the precursor material by the presence of the inert gas.

9. The method of claim 1, wherein the inert gas is nitrogen.

10. The method of claim 1, wherein the precursor material is formed into a shape having the imparted geometry and pattern thereon after laser cutting thereof.

11. The method of claim 1, wherein the precursor material is a tube having the imparted geometry and pattern thereon after laser cutting thereof.

12. The method of claim 1, wherein providing the precursor material further comprises providing a drug or bio-active agent in or on some or all of the precursor material prior to laser cutting.

13. The method of claim 12, wherein the drug or bio-active agent comprises 1-50%, and preferably 10-30%, weight of the device.

14. The method of claim 1, further comprising providing a drug or bio-active agent in or on some of the precursor material after laser cutting thereof has occurred.

15. The method of claim 1, further comprising providing a radiopaque material in or on some or all of the precursor sheet prior to laser cutting thereof.

16. The method of claim 1, further comprising providing a radiopaque material in or on some or all of the precursor material after laser cutting thereof.

17. The method of claim 1, wherein imparting the geometry and pattern comprises imparting a helical design to the precursor material by the laser cutting thereof.

18. The method of claim 1, wherein imparting the geometry and pattern comprises imparting a non-helical design to the precursor material by the laser cutting thereof.

19. The method of claim 1, wherein imparting the geometry and pattern comprises imparting a combination of a helical and a non-helical design to the precursor material by the laser cutting thereof.

20. The method of claim 1, wherein imparting the geometry and pattern comprises imparting the geometry and pattern over one of an entire length of the intraluminal medical device, a portion of the entire length thereof, or at intervals along the entire length thereof.

21. The method of claim 1, wherein the device is a stent.

22. The method of claim 13, wherein the % weight of the drug or bio-active agent is substantially unaffected by the laser cutting of the precursor material.

23. An intraluminal medical device comprising:
a bioabsorbable precursor material having a geometry or pattern imparted thereto by laser cutting in the presence of an inert gas;
at least one drug or bio-active agent incorporated into or onto the device; and
at least one radiopaque material incorporated into or onto the device.

24. The intraluminal medical device of claim 23, wherein the precursor material is a sheet formed into a shape for intraluminal receipt after the geometry or pattern is imparted thereto.

25. The intraluminal medical device of claim 23, wherein the precursor material is a tube.

26. The intraluminal medical device of claim 23, wherein the geometry or pattern is a helical design.

27. The intraluminal medical device of claim 23, wherein the geometry or pattern is a non-helical design.

28. The intraluminal medical device of claim 23, wherein the non-helical design is a series of longitudinally adjacent segments.

29. The inraluminal medical device of claim 23, wherein the geometry or pattern is a combination of helical and non-helical designs.

30. The intraluminal medical device of claim 23, wherein the geometry or pattern extends wholly, partially, or at discrete segments of a length of the device.

31. The intraluminal medical device of claim 23, wherein the at least one drug or bio-active agent is provided between 1-50% by weight.

32. The intraluminal medical device of claim 31, wherein the at least one drug or bio-active agent is provided between 10-30% by weight.

33. The intraluminal medical device of claim 31, wherein the % weight of the at least one drug or bio-active agent is substantially unaffected by the laser cutting of the device.

34. The intraluminal medical device of claim 22, wherein the device is a stent.
